(19) United States
(12) Patent Application Publication
(10) Pub. No.: US 2004/0208842 A1
(43) Pub. Date: Oct. 21, 2004

(54) Antimicrobial cleansing compositions and methods of use

(76) Inventors: Branson W. Ritchie, Athens, GA (US); Douglas T. Kemp, Watkinsville, GA (US)

Correspondence Address: WOMBLE CARLYLE SANDRIDGE & RICE, PLLC, P.O. BOX 7037, ATLANTA, GA, 30357-0037, US

(21) Appl. No.: 10/812,668

(22) Filed: Mar. 30, 2004

Related U.S. Application Data
(63) Continuation-in-part of application No. 10/739,841, filed on Dec. 18, 2003.
Application No. 10/739,841 is a continuation-in-part of application No. 09/955,657, filed on Sep. 18, 2001.

(51) Int. Cl. A61K 7/075; A61K 7/08

(52) U.S. Cl. 424/70.21

(57) ABSTRACT

The present invention provides a skin cleanser comprising a chelating agent, a buffering agent, a carrier and a detergent such as cocamidopropyl betaine and at concentrations that avoid substantial irritation of the skin while having the ability to cleanse the skin surface of contaminants. The compositions are especially useful to cleanse the external ear canal of an animal. The chelating agent has a synergistic effect on the ability of the detergent to loosen cerumen and other contaminants from the skin surface, and also on the antimicrobial and antifungal activity of the detergent, thereby reducing microbial colonization that may develop into a pathological condition if not treated. The present invention provides methods for cleansing a skin surface by using the ccleanser which may include more than one antimicrobial agent to inhibit the proliferation of a single invasive organism, or a mixed population of invasive organisms. The invention also provides kits that have packaging that includes instructions on how to prepare and use the compositions to cleanse the surface of the skin.

The present invention is a continuation-in-part of non-provisional U.S. application Ser. No. 10/739,841, filed Dec. 18, 2003, which is incorporated herein by reference in its entirety.

### FIELD OF THE INVENTION

The present invention relates generally to methods and compositions for cleansing a surface. The present invention further relates to compositions and methods for the cleansing and sanitizing of the skin surface of an animal or human.

### BACKGROUND

The outermost layers of the skin form a physical barrier that protects an animal from microbial invasion and the establishment of opportunistic infections.

Injuries to the skin, for instance an abrasion, incision, laceration, a burn from thermal, radiant or chemical exposure or a necrotic lesion of the surface tissue, destroy the integrity of the cornified layer, epidermis or dermis and will allow microbes to penetrate into the underlying tissues. An established infection can become systemic and ultimately life-threatening.

Otitis externa is an inflammation, typically infective, of the external auditory canal, which is an extension of the skin. A microbial population can colonize the surface of the auditory canal, especially by proliferating in the cerumen, the waxy secretion of the ear. Breakdown of the cerumen and of dead microorganisms will often lead to malodor and discharge from the ear. Inflammation will often follow. In animals such as dogs, the first sign of otitis externa is most likely to be the smell, and shaking of the animal's head. The development of pain sensitivity can cause the animal to tilt its head and to resist touch.

Otitis externa is often initiated by minor injury to the dermal layers such as from scratching, often combined with a reduction or loss of the lipid layer over the skin surface. Bacteria may then enter the underlying tissues of the ear canal and establish an infection. The most common bacteria involved are Pseudomonas spp, Staphylococci spp., E. coli and Proteus spp. Fungi, especially Aspergillus spp., Candida spp. and Malassezia spp. are also often present. In severe cases, there can be spreading to the pinna and the temporal bone adjacent to the ear canal and to the inner ear, causing hearing loss.

Common treatments for otitis externa begin with mechanical cleaning of the external ear canal to remove any discharge, waxy deposits or other detritus. Antimicrobials are then applied, often as ear drops that contact the surface of the skin providing that the surface has been adequately cleared of the cerumen. As with all treatments requiring antimicrobial use, there is the undesirable possibility of developing or promoting resistant strains of pathogens.

Besides the common problem of otitis externa in dogs, as well as in other animals and humans, microbial colonizations can be found on any animal skin surface and will require similar treatment regimens.

Sanitization can be necessary when a skin surface has contacted a contaminated surface. For example, bare feet contacting soil or a frequently traveled floor area may need to be cleansed to avoid an infection such as athlete's foot. Sanitizing foot washes are often found leading to swimming areas. Environmentally acceptable cleansers and sanitizers are also in demand in a medical facility to clean exposed surfaces, instruments and patient skin.

What is needed, therefore, are cleansers with components that enhance antimicrobial activity thereby allowing decreased amounts of the effective antimicrobial agents and detergents compared to cleansers that lack the enhacers. What is needed are cleansers, and methods of use, that will cleanse and/or sanitize a skin surface without causing unacceptable irritation. The cleansers should also discourage the development or proliferation of resistant microorganisms and be minimally injurious environmentally.

In particular, there is a need not addressed in the prior art for compositions and methods of their use that will effectively clean the external ears of an animal of waxy deposits and the microbial populations that are proliferating in such deposits. Suitable compositions should have enhanced antimicrobial activity that lessens the effective concentrations of antimicrobials necessary, and which do not further irritate or inflame a treated ear. Since otitis externa is often accompanied by additional wounds to the skin surface which may be, or become, infected, the compositions may have wound treating activity to promote healing and pain relief.

There is also a need, therefore, not addressed in the prior art, for compositions to treat infection-susceptible skin wounds of humans and animals and which may also promote wound healing. Such methods may also offer relief from pain and provide protection from infection. Safe and effective means of topically administering such compositions to a skin surface are also desirable. Suitable compositions preferably would also have enhanced activity against drug resistant strains of infectious microbes compared to antimicrobial compositions not including a chelating agent.

### SUMMARY OF THE INVENTION

The present invention addresses the need to cleanse a skin surface of an animal or human patient. In particular, the invention addresses the need for effective methods to cleanse the ear in animal and human patients with otitis externa, staphylococcal dermatitis or fungal dermatitis. Briefly described, the present invention provides methods, and compositions for use in the method, for cleansing of skin or other surfaces. The skin cleansers of the invention comprise a chelating agent, a pH buffer, a detergent, and optionally, an antimicrobial agent, and a carrier. The detergent and antimicrobial agent(s) each have increased antimicrobial activity because of the synergy with the chelating agent and maintenance of the treated area at a pH suitable for sustained antibiotic activity. The detergent and antimicrobial agent can, therefore, be used in effective doses that are less than would be required for the same level of antimicrobial activity in the absence of the chelator. The compositions of the present invention are particularly effective against skin colonizations caused by drug-resistant strains of microbes.

Accordingly, one aspect of the present invention is a skin cleanser composition comprising a chelating agent, a buffering agent, and a detergent. In one embodiment, the detergent is a mild detergent that will avoid irritation to the surface of the skin or other tissue to which the composition is applied. For instance, one suitable detergent is cocamidopropyl betaine, but it is contemplated that any detergent known to those of ordinary skill and which will clean a wound or skin surface without triggering an inflammatory reaction or otherwise further extends the extent of injury of the recipient patient can be used.

Another aspect of the present invention provides methods for cleansing a skin surface wherein the skin of a human or animal is contacted with a skin cleanser comprising a chelating agent, a buffer, optionally an antimicrobial agent, a detergent and a carrier, wherein the amounts of the chelating agent and the detergent are selected to allow the chelating agent and the detergent to synergistically cooperate to enhance antimicrobial activity of the skin cleanser when in aqueous solution. More than one antimicrobial agent may be used to inhibit the proliferation of a single invasive organism, or a mixed population of invasive organisms. The antimicrobial agent(s) can be selected after determining the composition and antibiotic resistance spectrum of the invading microbial population.

The skin cleanser of the invention can be applied to any skin surface. The components of the skin cleanser, when prepared for application, have concentrations that avoid substantial irritation of the skin while having the ability to cleanse the skin surface of contaminants. In particular, the methods and compositions of the invention are especially useful to cleanse the external ear canal of, for example, a dog. Initially, the ear may be washed with a skin cleanser comprising a chelating agent, a pH buffer and a detergent with, preferably, an aqueous carrier. The chelating agent will have a synergistic effect on the ability of the detergent to loosen cerumen and other contaminants from the skin surface, and also on the antimicrobial and antifungal activity of the detergent, thereby reducing microbial colonization that may develop into a pathological condition if not treated.

Another aspect of the invention provides kits that comprise a skin cleanser as described above, or the components to prepare the compositions, and packaging that includes instructions on how to prepare and use the compositions to cleanse the surface of the skin. One embodiment of the invention comprises a vessel containing a chelating agent, a pH buffering agent, a detergent and packaging material comprising instructions for using the kit for preparing the cleanser and delivering it to a skin surface of a human or animal to accelerate cleansing of the skin.

In other embodiments of the kits of the invention, the skin cleanser may further comprise an antimicrobial agent and additional instructions for using the antimicrobial agent with the skin cleanser to clean and sanitize a skin surface and, optionally, instructions for using the skin cleanser to reduce otitis externa of an animal or human. In yet other embodiments of the invention, the kit may include a medical dressing configured to receive the cleanser and instruction for using the medical dressing to deliver the cleanser to the skin surface of an animal or human.

Additional objects, features, and advantages of the invention will become more apparent upon review of the detailed description set forth below when taken in conjunction with the accompanying drawing figures, which are briefly described as follows.

### BRIEF DESCRIPTION OF THE FIGURES

FIG. 1. Isobologram, illustrating the combined effect of EDTA and neomycin (in 50 mM Tris) on Staphylococcus aureus.

FIG. 2. Isobologram, illustrating the combined effect of EDTA and neomycin (in 50 mM Tris) on Pseudomonas aeruginosa.

FIG. 3. Isobologram, illustrating the combined effect of EDTA and neomycin (in 50 mM Tris) on Enterococcus faecalis.

FIG. 4. Growth of Staphylococcus aureus on Mueller Hinton agar when treated alone or with combinations of EDTA, water, and neomycin.

FIG. 5. Growth of Pseudomonas aeruginosa on Mueller Hinton agar when treated alone or with combinations of EDTA, water, and neomycin.

FIG. 6. Growth of Enterococcus faecalis on M Enterococcus agar when treated alone or with combinations of EDTA, water, and neomycin.

### DETAILED DESCRIPTION OF THE INVENTION

A full and enabling disclosure of the present invention, including the best mode known to the inventor of carrying out the invention is set forth more particularly in the remainder of the specification, including reference to the Examples. This description is made for the purpose of illustrating the general principles of the invention and should not be taken in the limiting sense.

The present invention addresses the need to effectively remove contaminants, such as cerumen, skin detritus, and colonizing bacteria and fungi from the surface of an animal skin. The present invention addresses the need to cleanse the external ear surfaces of an animal, to reduce or eliminate otitis externa, a common complaint of deistic dogs. The present invention, therefore, provides skin cleanser compositions that comprise a chelating agent, a buffer and a detergent that, when applied to the skin surface in a carrier, will effectively loosen contaminants, expecially cerumen, from the skin. The compositions may further comprise antimicrobial agents and an anti-inflammatory agent. The detergent or antimicrobial agent(s) has increased antimicrobial activity because of the synergy with the chelating agent and maintenance of the treated area at a pH suitable for sustained antibiotic activity. The antimicrobial agent can, therefore, be used in effective doses that are less than would be required for the same level of antimicrobial activity in the absence of the chelator.

The compositions of the present invention are, therefore, useful in counteracting or preventing an infection or will be more effective against infections caused by drug-resistant strains of microbes.

The present invention further provides methods for cleansing the surface of an animal's skin of contaminants. The methods are particularly useful to inhibit or reduce the microbial colonization of a skin surface such as the external ear of an animal.

Definitions

The term “skin” as used herein refers to any exterior cornified surface of the epidermal layer of an animal, including the external ear canal leading to the tympanum. The skin may be intact or have a wound.

The term “wound” as used herein refers to a lesion or open wound that can expose underlying epidermal, dermal, muscular or adipoidal tissue to the air. Wounds include, but are not limited to, an abrasion, a puncture wound, an incision, a laceration, a penetrating wound, a perforating wound, a tunnel wound and the like. Wounds also include open wounds that have been sutured or otherwise mechanically closed but have not healed or repaired the break in the skin.

The terms “lesion” and “surface lesion” as used herein refer to a circumscribed area of pathologically altered tissue, an injury or wound. Primary lesions are the immediate result of the pathological condition and include, but are not limited to, cuts, abrasions, vesicles, blebs, bullae chancres, pustules, tubercles or any other such condition of the skin or a surface of the mouth, nose, anus or any other orifice of the body of a human or animal, or to the surface layers of the eye including the conjunctiva and cornea, or secondary lesions that later develop from a primary lesion and includes, but is not limited to, fissures and ulcers and other wounds.

The terms “wound healing” and “wound repair” refer to a process involving tissue growth that partially or totally closes a wound, repairs a breach in the dermis or epidermis and partially or totally restores the barrier properties of the skin or the repair of the surface layers of the eye including the conjunctiva and cornea. The term “microbial infection” as used herein refers to any pathological presence of at least one bacterial species on or in an injury or lesion to the skin of a human or animal.

The term “colonization” as used herein refers to a microbial (bacterial or fungal) population residing on a skin surface. The population may be pathological, thereby forming an infection, or non-pathological. A microbial colonization may comprise the normal flora typically found on the skin surface of the subject animal or an abnormal or a typical colonization with no overt symptoms of presence or producing indications of presence, such as by emitting an odor, without causing a pathology of the skin.

The term “antimicrobial agent” as used herein refers to the compounds and combinations thereof, including bacteristatic or bactericidal, antifungal, compositions or agents, that may be administered to an animal or human and which inhibit the proliferation of a microbial population.

The term “chelating agent” as used herein refers to any organic or inorganic compound that will bind to a metal ion having a valence greater than one.

The term “pH buffering agent” as used herein refers to any organic or inorganic compound or combination of compounds that will maintain the pH of a detergent and/or antibiotic-containing solution within 0.5 pH units of a selected pH value.

The term “carrier” as used herein refers to any solvent of antibiotics, chelating agents and pH buffering agents that will allow a skin cleanser composition to be topically administered directly to the skin. The carrier will also allow a composition to be applied to a medical dressing for application to such a wound. A “carrier” as used herein, therefore, refers to such solvent as, but not limited to, water, saline, physiological saline, ointments, creams, oil-water emulsions, gels, or any other solvent or combination of solvents and compounds known to one of skill in the art that is pharmaceutically and physiologically acceptable to the recipient human or animal.

The term “skin cleanser” as used herein refers to a composition that when applied to a skin surface will desquamate, defoliate, or loosen lipids, lipid-like material, cerumen and other deposits or contaminants on the skin surface.

The term “sanitization” as used herein refers to the process of reducing or removing a microbial population from the surface of the skin. A sanitized skin surface may be completely free of microbes (sterile), or have a reduced microbial population compared to the microbial population before sanitization.

The term “contaminants” as used herein refers to any undesirable material, particle or organism on the surface or within strata of the cornified skin layer. Examples of contaminants include, but are not limited to, bacteria, fungi, fungal spores, soil, grease or oil (produced by the skin including sebaceous secretions, cerumen and the like).

The term “synergistically cooperate” as used herein refers to the interaction between a chelating agent and a detergent and/or an antimicrobial agent whereby the antimicrobial activity of a combination of a chelator and detergent and/or antimicrobial agent is greater than the sum of the individual activities of the chelator, detergent or antimicrobial agent.

One aspect of the present invention provides methods for cleansing a skin surface wherein the skin of a human or animal is contacted with a skin cleanser comprising a chelating agent, a buffer, optionally an antimicrobial agent, a detergent and a carrier. More than one antimicrobial agent may be used to inhibit the proliferation of a single invasive organism, or a mixed population of invasive organisms. The antimicrobial agent(s) can be selected after determining the composition and antibiotic resistance spectrum of the invading microbial population.

The skin cleanser of the invention can be applied to any skin surface. The components of the skin cleanser, when prepared for application, have concentrations that avoid substantial irritation of the skin while having the ability to cleanse the skin surface of contaminants. In particular, the methods and compositions of the invention are especially useful to cleanse the external ear canal of, for example, a dog. Initially, the ear may be washed with a skin cleanser comprising a chelating agent, a pH buffer and a detergent with, preferably, an aqueous carrier. The chelating agent will have a synergistic effect on the ability of the detergent to loosen cerumen and other contaminants from the skin surface, and also on the antimicrobial and antifungal activity of the detergent, thereby reducing microbial colonization that may develop into a pathological condition if not treated.

It is also contemplated for the cleansers to be applied to the hair or fur of an animal such that undesired dirt particles skin debris and other contaminates may be removed. When applied to the hair or fur of an animal, the compositions of the invention are also useful to inhibit or eliminate a fungal or bacterial colonization or infection of the hair strands. The cleanser, when applied to the hair may also contact the skin, whereupon the cleanser may inhibit or eliminate an infection that is on both the hair strand and extending into or adjacent to the hair follicle.

The antimicrobial activity of the skin cleanser can be enhanced by including one or more antimicrobial agents such as an antibacterial antibiotic for an antifungal agent or combinations thereof. The chelating agent of the skin cleanser will also synergistically enhance the activity of the antimicrobial agents, as shown in Examples 1-5 below. The antimicrobial agent can be applied to the skin surface by combining with the skin cleanser that includes the detergent, or after application of the skin cleanser to dislodge and clear the surface of lipid and lipid-like contaminants. By following application of the skin cleanser, microbial populations are more readily exposed to the antimicrobial agent(s). When applied to the skin other than in the skin cleanser, the antimicrobial agent(s) is combined with a chelating agent that synergistically enhances the antimicrobial activity.

It is further contemplated that the skin surface may also be treated with a wound management composition that also has antimicrobial activity and wound healing properties. A suitable composition, and methods of use thereof, for use with the compositions and methods of the present invention are fully described in U.S. patent application Ser. No. 10/739,841 incorporated herein by reference in its entirety.

The skin surface may be washed with the skin cleanser before the application of antimicrobial composition using a skin cleanser comprising a chelating agent having a concentration from about 1 mM to about 250 mM, a buffering agent having a concentration of about 10 mM to about 250 mM and a detergent having a concentration from about 1 to about 30%, by volume, as given, for instance, in Example 15 below. Typically, a carrier such as an aqueous carrier, may be included in the corporation to dissolve the components if not soluble in the detergent. An aqueous environment is also preferred for the effective buffering and chelation by the skin cleanser. In one embodiment, the composition comprises about 8 mM EDTA and 20 mM Tris and about 10%, by volume, of cocamidopropyl betaine

The skin cleanser used in the methods herein described may be applied to a skin surface by any number of methods, including as a lavage where the skin is washed or irrigated. The methods of the invention are particularly useful in cleansing the external auditory canal of a human or animal. In this case, the canal may be flooded with the skin cleanser, for example, by using a syringe, the nipple of which can be inserted directly into the canal. The skin cleanser can also be delivered to the ear canal over a prolonged period using a wick. The skin cleanser can be absorbed onto the surface of the fibers of a wound dressing before or during the treatment, ensuring that while the wound is ventilated it is still subject to contact with the compositions for a prolonged period. The skin cleanser compositions of the present invention may also be used as a bath for the total or partial immersion of a human or animal for the cleansing of part or all of the surface of a human or animal.

The chelating agent of the compositions of the present methods may be selected from ethylenediamenetetracetic acid (EDTA), triethylene tetramine dihydrochloride (TRIEN), ethylene glycol-bis (beta-aminoethyl ether)-N, N, N′, N′-tetracetic acid (EGTA), diethylenetriamin-pentaacetic acid (DPTA), triethylenetetramine hexaacetic acid (TTHA), deferoxamine, Dimercaprol, edetate calcium disodium, zinc citrate, penicilamine succimer and Editronate or any other chelating agent, salt or combination thereof, known to one of ordinary skill in the art, and which will chelate divalent metal ions such as, but not only, Ca²⁺, Mg²⁺, Mn²⁺ Fe²⁺, and Zn²⁺. Preferably, the chelating agent selected will not irritate the skin when at the concentration typically applied. The chelating agent, when delivered to the skin of a human or animal patient will have a concentration between from about 1 mM to about 250 mM, more preferably from about 1 mM to about 100 mM, most preferably from about 1 mM to about 50 mM. In a preferred embodiment, the chelating agent is EDTA at a concentration of about 8 mM.

The compositions of the present invention also include a pH buffer that preferably will maintain the pH of the skin cleanser, when delivered to the skin, to between about pH 7.0 and about pH 9.0. A pH buffer may be selected from, but is not limited to, Tris (hydroxymethyl) aminomethane (tromethaprim; TRIZMA base), or salts thereof, phosphates or any other buffering agent such as, for example, phosphate-buffered saline that is biologically acceptable. When Tris base is the selected pH buffer, the pH may be adjusted by the addition of hydrochloric acid, thereby forming Tris-HCl. In a preferred embodiment, the pH of the antimicrobial composition in solution is about 8.0. The buffer, when delivered to a wound, has an effective dose of between about 5 mM and about 250 mM, more preferably between about 5 mM and about 100 mM, most preferably between about 10 mM and about 100 mM. In a preferred embodiment the buffer agent has a concentration of about 20 mM.

The compositions of the present invention may also comprise at least one antimicrobial agent. The infections that may be treated by the methods and compositions of the present invention may be any opportunistic infection of a wound by a bacterium, or a multiple infection of more than one species of bacteria. The skin cleansers of the present invention are also useful to sanitize a skin surface colonized by a microbial population. Microbial species that may cause infections inhibited by the methods of the present invention include fungi and bacterial species that may cause infections of a lesion, or other break in the skin of a human or animal including, but are not limited to, Aerobacter aerongenes, Aeromonas spp., Bacillus spp., Bordetella spp, Campylobacter spp., Chlamydia spp., Corynebacterium spp., Desulfovibrio spp., Escherichia coli, enteropathogenic E. coli, Enterotoxin-producing E coli, Helicobacter pylori, Klebsiella pneumoniae, Legionella pneumophiia, Leptospira spp., Mycobacterium tuberculosis, M. bovis, Neisseria gonorrhoeae, N. meningitidis, Nocardia spp., Proteus mirabilis, P vulgaris, Pseudomonas aeruginosa, Rhodococcus equi, Salmonella enteridis, S. typhimurium, S. typhosa, Shigella sonnei, S dysenterae, Staphylococcus aureus, Staph. epidermidis, Streptococcus anginosus, S. mutans, Vibrio cholerae, Yersinia pestis, Y. pseudotuberculosis, Actinomycetes spp., and Streptomyces spp.

The action of the antimicrobial agent can be either bacteriostatic where the antibiotic arrests the proliferation of, but does not necessarily kill, the microorganism, the antibiotic activity can be bacteriocidal and kill the organism or a combination of activities. Antibiotics suitable for use in the compositions and methods of the present invention include, but are not limited to, β-lactams (Penicillins and cephalosporins), vancomycins, bacitracins, macrolides (erythromycins), lincosamides (clindomycin), chloramphenicols, tetracyclines, aminoglycosides (gentamicins), amphotericns, cefazolins, clindamycins, mupirocins, sulfonamides and trimethoprim, rifampicins, metronidazoles, quinolones, novobiocins, polymixins and Gramicidins and the like and any salts or variants thereof. It also understood that it is within the scope of the present invention that the tetracyclines include, but are not limited to, immunocycline, chlortetracycline, oxytetracycline, demeclocycline, methacycline, doxycycline and minocycline and the like. It is also further understood that it is within the scope of the present invention that aminoglycoside antibiotics include, but are not limited to, gentamicin, amikacin and neomycin and the like.

In various embodiments, the antibiotic is a penicillin, an aminoglycoside, a vancomycin, a chloramphenicol, an erythromycin, a tetracycline, gentamicin, nalidixic acids, or a streptomycin. In another embodiment the antibiotic is tetracycline. In a preferred embodiment of the present invention, the antibiotic is neomycin. In another embodiment of the present invention, the antibiotic is amikacin. In yet another embodiment, the antibiotic is gentamicin. However, a combination of antibiotics may be selected and used depending upon the antibiotic resistance profiles of the microbial population of the wound.

Techniques to identify the infecting microorganism and to determine the concentration of the antibiotic that will inhibit or kill fifty percent (MIC₅₀) of the organisms will be considered well known to one of ordinary skill in the art and will not require undue experimentation. Such procedures are especially useful if, for example, an animal has a prolonged and persistent ear infection or colonization. It is advantageous to select an antimicrobial agent more specifically directed to the identified target microbial species. The techniques to determine the antibiotic sensitivity of a bacterial isolate, and the methods of determining the synergistic effect of adding a chelating agent to a solution of an antibiotic are described in Manual of Methods for General Microbiology, Eds. Gerhardt et al., American Society of Microbiology, 1981, and incorporated herein in its entirety by reference.

The concentrations and amounts of the antimicrobial agent and chelating agent may be adjusted to levels that are physiologically accepted by exposed tissue of an injury or lesion and effective against the microbial population of the skin. In one embodiment of the present invention, the concentration of the antibiotic is in the range of about 0.04 mg/ml to about 25 mg/ml and the concentration of the chelating agent in the carrier is in the range of about 0.1 mM to about 100.0 mM.

The compositions for use in the methods of skin cleansing may also comprise a surfactant for cleaning the skin, or contributing to bactericidal activity of the administered compositions. Preferably, the detergent selected for application to a wound or skin surface is mild and will not lead to extensive irritation or promote further tissue damage to the patient.

Suitable nonionic surfactants which can be used are, for example: fatty alcohol ethoxylates (alkylpolyethylene glycols); alkylphenol polyethylene glycols; alkyl mercaptan polyethylene glycols; fatty amine ethoxylates (alkylaminopolyethylene glycols); fatty acid ethoxylates (acylpolyethylene glycols); polypropylene glycol ethoxylates (Pluronic); fatty acid alkylolamides (fatty acid amide polyethylene glycols); alkyl polyglycosides, N-alkyl-, N-alkoxypolyhydroxy fatty acid amide, in particular N-methyl-fatty acid glucamide, sucrose esters; sorbitol esters, and esters of sorbitol polyglycol ethers. In one embodiment, the surfactant is polypropylene glycol ethoxylate. In another embodiment, the detergent is cocamidopropyl betaine at a final concentration, when applied to a skin surface, of between about 1% and 25%, by volume, most preferably at about 10%, by volume.

The skin cleanser compositions for use in the methods of the invention preferably include a carrier that provides the medium in which are dissolved or suspended the constituents of the compositions. Suitable carriers include any aqueous medium, or an oil, emulsion, ointment and the like that will allow the compositions to be delivered to the skin without irritating the surface, and which can be readily rinsed to remove skin contaminants. A preferred carrier is an aqueous medium such as, but not limited to, saline or water.

It is also contemplated that the skin cleansers of the invention can be prepared as a precursor dry preparation, solution, or as concentrate that is useful for the extemporaneous preparation of the administered compositions. In one embodiment of the invention, the Tris and EDTA are provided combined as a dry composition that can be mixed with detergent such as, but not limited to, cocamidopropyl betaine. In various embodiments, the Tris and EDTA are suspended in a detergent, such as cocamidopropyl betaine, before adding an aqueous carrier. Optionally, the compositions may further include a stabilizer to extend the shelf-life of the composition. A particularly useful stabilizer is scorbic acid, preferably as the sodium or potassium salt.

Medical dressings suitable for use in the methods of the present invention for contacting a skin surface with the skin cleanser compositions can be any material that is biologically acceptable and suitable for placing over any wound or surface of the skin. In exemplary embodiments, the support may be a woven or non-woven fabric of synthetic or non-synthetic fibers, or any combination thereof. The dressing may also comprise a support, such as a polymer foam, a natural or man-made sponge, a gel or a membrane that may absorb or have disposed thereon, a therapeutic composition. A gel suitable for use as a support for the antimicrobial composition of the present invention is KY™ (sodium carboxymethylcellulose 7H 4F (Hercules, Inc., Wilmington, Del.)).

The present invention also contemplates that a gauze may be impregnated with the skin cleanser composition and then dried. This allows the impregnated dressing to be stored for later use, or to avoid excessively dampening an injured area. In yet another embodiment of the present invention, a skin cleanser composition is absorbed on the surface of the support material of the medical dressing. The composition may be applied to the surface by wetting the surface with a solution of the skin cleanser and drying the support to deposit the composition thereon. A concentration of the composition that is effective for cleansing and/or sanitizing the skin surface may be attained when the dressing is wetted by the patient's body.

Another aspect of the present invention is a skin cleanser composition comprising a chelating agent, a buffering agent, and a detergent. In one embodiment, the detergent is a mild detergent that will avoid irritation to the surface of the skin or other tissue to which the composition is applied. For instance, one suitable detergent is cocamidopropyl betaine, but it is contemplated that any detergent known to those of ordinary skill and which will clean a wound or skin surface without triggering an inflammatory reaction or otherwise further extends the extent of injury of the recipient patient can be used. In one embodiment, the detergent is about 3% to about 33%, by volume, of a therapeutic composition. In a preferred embodiment, the detergent is about 10%, by volume, of cocamidopropyl betaine.

In various embodiments of the invention, the composition further comprises an antimicrobial agent and optionally a carrier. The embodiments may also include a preservative that will increase the shelf-life of the compositions. A typical preservative is scorbic acid, or the salts thereof. The various embodiments of the compositions can further comprise an anti-inflammatory agent such as, but not limited to dexamethasone.

In the compositions of the present invention, the chelating agent is selected from the group consisting of ethylenediamenetetracetic acid (EDTA), triethylene tetramine dihydrochloride (TRIEN), ethylene glycol-bis (beta-aminoethyl ether)-N, N, N′, N′-tetracetic acid (EGTA), diethylenetriamin-pentaacetic acid (DPTA), triethylenetetramine hexaacetic acid (TTG), deferoxamine, Dimercaprol, edetate calcium disodium, zinc citrate, penicilamine succimer and Editronate. Preferably, the chelating agent is ethylenediamenetetracetic acid (EDTA).

The antimicrobial agent(s) that may be included in the various embodiments of the compositions include, but are not limited to, a β-lactam, an aminoglycoside, a vancomycin, a bacitracin, a macrolide, an erythromycin, a lincosamide, a chloramphenicol, a tetracycline, a gentamicin, an amphotericin, a cefazolin, a clindamycin, a mupirocin, a nalidixic acid, a sulfonamide and trimethoprim, a streptomycin, a rifampicin, a metronidazole, a quinolone, a novobiocin, a polymixin and a gramicidin. More preferably, the antibiotic s selected from the group consisting of a β-lactam, an aminoglycoside, a vancomycin, a chloramphenicol, an erythromycin, a tetracycline, gentamicin, nalidixic acid and a streptomycin. In one embodiment, the antimicrobial agent is oxytetracycline. In another embodiment, the antimicrobial agent is amikacin. In yet another embodiment, the antimicrobial agent is neomycin.

In various embodiments of the compositions, the pH buffer can be Tris (hydroxymethyl) aminomethane (TRIZMA base) which, when dissolved in a carrier with a suitable acid such as HCl, will have a concentration of between about 5 mM and about 250 mM, preferably between about 5 mM and about 100 mM, more preferably between about 10 mM and about 100 mM. In one embodiment, the concentration of the buffering agent is about 20 mM. In the embodiments of the compositions, the cleanser can have a pH of between about 6.0 and about 8.0. In one embodiment the pH is about 7.4.

Another aspect of the invention is kits that comprise a skin cleanser as described above, or the components to prepare the compositions, and packaging that includes instructions on how to prepare and use the compositions to cleanse the surface of the skin. One embodiment of the invention, therefore, comprises a vessel containing a chelating agent, a pH buffering agent, a detergent and packaging material, the packaging material comprising instructions directing the use of the kit for preparing the composition of the present invention and delivering the composition to a skin surface of a human or animal to accelerate cleansing of the skin.

In another embodiment of the kit of the invention, the skin cleanser further comprises an antimicrobial agent and further instructions for using the antimicrobial agent with the skin cleanser to clean and sanitize a skin surface.

Another embodiment of the kit of the invention further comprises instructions for using the skin cleanser to reduce otitis externa of an animal or human.

Yet another embodiment of the invention, the kit further comprises a medical dressing configured to receive the cleanser and instruction for using the medical dressing to deliver the cleanser to the skin surface of an animal or human.

Even though the invention has been described with a certain degree of particularity, it is evident that many alternatives, modifications, and variations will be apparent to those skilled in the art in light of the present disclosure. Accordingly, it is intended that all such alternatives, modifications, and variations that fall within the spirit and the scope of the invention be embraced by the defined claims.

The following examples are presented to describe preferred embodiments and utilities of the present invention, but should not be construed as limiting thereof.

### EXAMPLE 1

### Determination of Synergistic Actions and Fractional Inhibitory Concentration (FIC) Index

The antibacterial action of combinations of EDTA-Tris and neomycin was measured by a two-dimensional microtiter checkerboard technique described in Gilman et al., The Pharmacological Basis of Therapeutics, eds Goodman and Gilman, 1085-1086 (Macmillan Publishing Co., New York, 1985), Sabath, L. D, Antimicrob. Agents and Chem. 210-217. (1967) and Sparks et al., Vet. Res. Comm. 18, 241-249 (1994), incorporated herein by reference in their entireties.

Each well of a round-bottomed 96-well microtiter plate is inoculated with 0.05 ml of 2-fold dilutions of neomycin and EDTA in 50 mM Tris. Then 0.05 ml of an 18-hour old culture of a test organism, containing 10⁶ colony-forming units (CFU)/ml, was added to each well. Controls for the culture and media were included in each plate. Plates were covered and incubated at 37° Celsius for 18-24 hours.

Results are plotted as isobolograms for the determination of antagonistic, neutral or additive, or synergistic effects. To generate isobolograms, FICs of the two test solutions were plotted individually on the x-axis and y-axis to determine the effect of combining the two test solutions on bacterial growth. A line that curves away from the zero point and the coordinates indicates antagonism. A straight line indicates neutral or additive effects. Lines that curve toward the zero point and the coordinates are indicative of synergism if there is at least a 4-fold decrease in the MIC of each compound, when used in combination, as compared with the MIC of each test compound alone.

A numerical score or fractional inhibitory concentration (FIC) index was determined. The FIC index is equal to the sum of the values of FIC for the individual drugs:

$$FIC = \frac{MIC of Drug A with Drug B}{MIC of Drug A } + \frac{MIC of Drug B with Drug A}{MIC of Drug B }$$

An FIC index greater than 1.0 indicates an antagonistic interaction, an FIC index of 1.0 indicates addition, and an FIC index of less than or equal to 0.5 indicates synergism between the two test agents.

### EXAMPLE 2

### Inhibition of the Growth of Microorganisms Infecting Burns

The organisms of this study were isolated from human burn patients. They included strains of methicillin resistant Staphylococcus aureus, and vancomycin resistant strains of Pseudomonas aeruginosa and Enterococcus faecalis. The bacterial isolates were propagated in or on Brain Heart Infusion broth (BHI), Mueller-Hinton Broth (MHB), blood agar (BA), Mueller-Hinton agar (MHA), enterococcus agar (EA), or 2× nutrient agar (2×NA).

The EDTA-Tris treatment solutions are prepared from a stock solution containing 0.5 mols/l sodium EDTA and 1.0 mols/l Tris-HCl, pH 8.0. The treatment solutions contained 5 mM sodium EDTA and 50 mM Tris-HCl with or without the addition of of neomycin sulfate 1 mg/ml.

Antibiotic resistance profiles were determined by the disc diffusion method on MHA (5). Antibiotics tested included ampicillin (AM-10), chloramphenicol (C-30), ciprofloxacin (CIP-5), kanamycin (K-30), gentamicin (GM-10), nalidixic acid (NA-30), neomycin (N-30), streptomycin (S-10), sulfisoxazole (G-25), tetracycline (Te-30), and vancomycin (Va-30).

Minimal Inhibitory Concentrations (MICs) and Minimal Bactericidal Concentrations (MBCs) for EDTA-Tris and neomycin were determined by the broth-dilution microtiter method in MHB or BHI according to the method of Blair et al., Manual of Clinical Microbiology. p.307 (pub: Am. Soc. Microbiol. Williams and Wilkins, Baltimore 1970), incorporated herein by reference in its entirety.

### EXAMPLE 3

### In Vitro Effect of EDTA-Tris and Neomycin on Enterococcus faecalis, Pseudomonas aeruginosa, and Staphylococcus aureus

2×NA plates were swabbed with 200 ml of an overnight culture containing about 10⁷ colony-forming-units of a test organism. The plates were sampled with multipoint contactors as described in Wooley et al., Am. J. Vet. Res. 35, 807-810 (1974). Each multipoint contactor consisted of an array of 27 mm sewing needles mounted to an aluminum plate measuring 1 mm×50 mm×50 mm. The needles were set 3.5 mm apart. The multipoint contactors were sterilized by autoclaving. To collect samples, a multipoint contactor was touched to an overnight bacterial culture grown on 2×NA as described above. Replicate plates were then inoculated by lightly pressing the needles bearing the test bacteria onto either MHA plates, BA plates or EA plates for Ps. aeruginosa, Staph. aureus and Ent. faecalis respectively. The agar plates were incubated at 37° C. and colonies were counted at 24 hours and 48 hours.

Each strain of microorganism was tested on a control agar plate (plate 1), and on plates wherein the inoculated bacteria were covered with a sterile surgical gauze saturated with 7 ml of: 5 mM EDTA-Tris (plate 2); 5 mM EDTA-Tris and 1 mg/ml neomycin (plate 3); 1 mg/ml neomycin (plate 4); sterile water (plate 5). Samples were taken at 0 mins, and at 30 mins, 1 hour, 2 hours, 4 hours, 6 hours, 8 hours, and 24 hours of incubation.

### EXAMPLE 4

### The Antibiotic Resistance Profiles, MIC and MBC Values for test Strains of Staph. aureus, Ps. aeruginosa, and Ent. faecalis

The antibiotic resistance profiles and MIC values for test strains of Staph. aureus, Ps. aeruginosa, and Ent. faecalis are shown on Table 1.

TABLE 1
Antibiotic resistance profiles of Staphylococcus aureus,
Pseudomonas aeruginosa, and Enterococcus faecalis.

| | Antimicrobial Agents^{A} | | | | | | | | | | |
|---|---|---|---|---|---|---|---|---|---|---|---|
| | Am | C | Cip | Gm | K | NA | N | S | G | Te | Va |
| Staphylococcus aureus | R^{B} | I | R | S | R | R | R | S | S | S | S |
| Pseudomonas aeruginosa | R | R | I | I | R | R | R | R | R | R | R |
| Enterococcus faecalis | S | R | R | R | R | R | R | R | R | R | R |

^{A}Am = ampicillin;
C = chloramphenicol;
Cip = ciprofloxacin;
K = kanamycin;
Gm = gentamicin;
NA = nalidixic acid;
N = neomycin;
S = streptomycin;
G = sulfisoxazole;
Te = tetracycline;
Va = vancomycin;
^{B}R = resistant;
I = intermediate;
S = sensitive.

Fractional inhibitory concentrations (FICs) and isoboloGrams for the EDTA-Tris-neomycin combination to determine a synergistic, additive, or antagonistic reaction, as described in Example 1, were determined for Staph. aureus, Ps. aeruginosa, and Ent. faecalis. MIC and MBC values for concentrations of neomycin, ampicillin, chloramphenicol, amikacin and oxytetracycline and EDTA administered individually, and the FIC values for Staph. aureus, Ps. aeruginosa, and Ent. faecalis are shown in Table 2 (Columns 2 and 3). MIC values for mixtures of the above antibiotics and EDTA in the presence of each other are shown in Table 2 (Columns 4 and 5 respectively).

TABLE 2
Minimal Inhibitory Concentration (MIC) data concerning the
amounts (mM) of EDTA in 50 mM Tris and antibiotics
(mg/ml) when reacting alone and in combination against
Pseudomonas aeruginosa, Staphylococcus aureus,
and Enterococcus faecalis.
MIC

| Individually Administered | | | Co-administered | | |
|---|---|---|---|---|---|
| | Neomycin | EDTA | Neomycin + EDTA | | FIC |
| Ps. aeruginosa | 1.0 | 1.25 | 0.063 | 0.156 | 0.19 |
| Staph. aureus | 3.13 | 1.0 | 1.56 | 0.25 | 0.75 |
| Ent. faecalis | 3.13 | 15.63 | 1.17 | 1.88 | 0.5 |
| | Ampicillin | EDTA | Ampicillin + EDTA | | FIC |
| Ps. aeruginosa | 0.49 | 1.25 | 0.123 | 0.156 | 0.38 |
| Staph. aureus | 0.24 | 1.0 | 0.0075 | 0.25 | 0.28 |
| Ent. faecalis | 0.001 | 15.63 | 0.00025 | 7.82 | 0.75 |
| | | | Chloramphenicol + | | |
| | Chloramphenicol | EDTA | EDTA | | FIC |
| Ps. aeruginosa | 12.5 | 1.25 | 1.56 | 0.313 | 0.37 |
| Staph. aureus | 0.39 | 1.0 | 0.39 | 1.0 | 2.0 |
| Ent. faecalis | 0.4 | 15.63 | 0.2 | 3.9 | 0.75 |
| | Amikacin | EDTA | Amikacin + EDTA | | FIC |
| Ps. aeruginosa | 0.001 | 1.25 | 0.001 | 1.25 | 2.0 |
| Staph. aureus | 0.12 | 1.0 | 0.03 | 0.5 | 0.75 |
| Ent. faecalis | 2.0 | 15.63 | 1.0 | 7.8 | 1.0 |
| | | | Oxytetracycline + | | |
| | Oxytetracycline | EDTA | EDTA | | FIC |
| Ps. aeruginosa | 0.003 | 1.25 | 0.00075 | 0.313 | 0.5 |
| Staph. aureus | 0.0001 | 1.0 | 0.00005 | 0.5 | 1.0 |
| Ent. faecalis | 0.05 | 15.63 | 0.025 | 3.91 | 0.75 |

*Synergistic reaction (FIC = approximately 0.5)
Additive reaction (FIC => .05 to approximately 1.0)
Antagonistic reaction (FIC => 1.0)

The MBC values for EDTA and neomycin were decreased by at least 75% for bacterial killing (MBC) in those situations in which synergistic potentation occurred (Ps. aeruginosa and Ent. faecalis) as shown in Table 3. A decrease of about 50% was observed with Staph. aureus.

TABLE 3
Minimal Bactericidal Concentrations (MBC), of Staphylococcus
aureus, Pseudomonas aeruginosa, and Enterococcus faecalis
reacted with EDTA (mM) and neomycin (mg/ml) in 50 mM Tris.

| Bacterial | | Individually | |
|---|---|---|---|
| Species | | Administered | Co-administered |
| Staph. aureus | EDTA (mM) | 7.81 | 3.9 |
| | Neomycin (mg/ml) | 3.13 | 1.56 |
| Ps. aeruginosa | EDTA (mM) | 250 | 20.0 |
| | Neomycin (mg/ml) | 5.0 | 0.04 |
| En. faecalis | EDTA (mM) | 250 | 62.5 |
| | Neomycin (mg/ml) | 25.0 | 6.25 |

Specifically in the case of Staph. aureus, the MBC values for EDTA and neomycin when combined were decreased by 50% as compared to the bactericidal effect of each when individually administered.

With Ps. aeruginosa, the MBC values for EDTA and neomycin when in combination were decreased 99.2% compared to when EDTA or neomycin were individually administered. In the case of Ent. faecalis, MBC values of EDTA and neomycin were both reduced 75% compared to when EDTA and neomycin were administered individually.

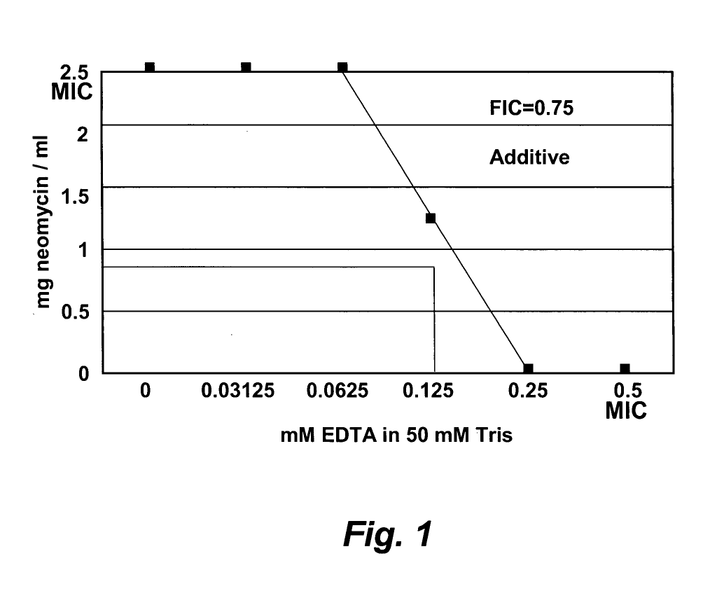

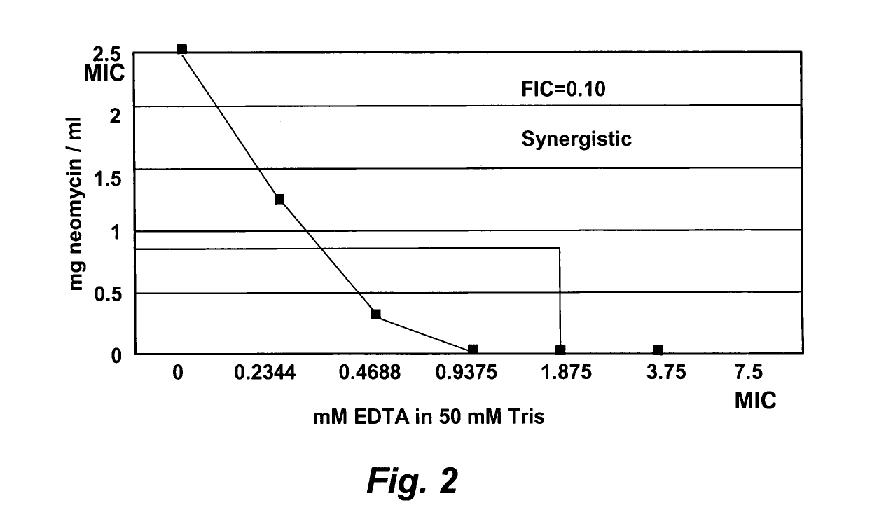

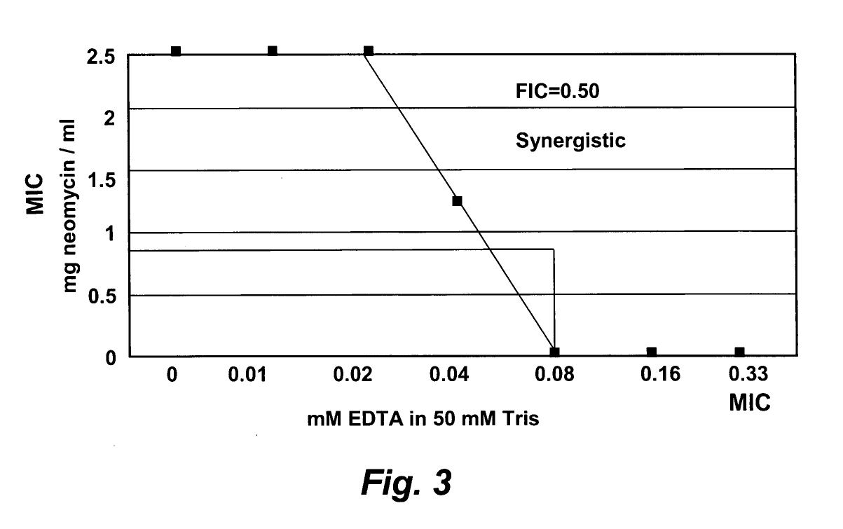

Synergistic effects were observed when various concentrations of EDTA-Tris and neomycin were reacted with Ps. aeruginosa and Ent. faecalis, while an additive effect was observed with Staph. aureus as shown in FIGS. 1-3.

### EXAMPLE 5

### Inhibition of Growth of Ps. aeruginosa and Staph. aureus

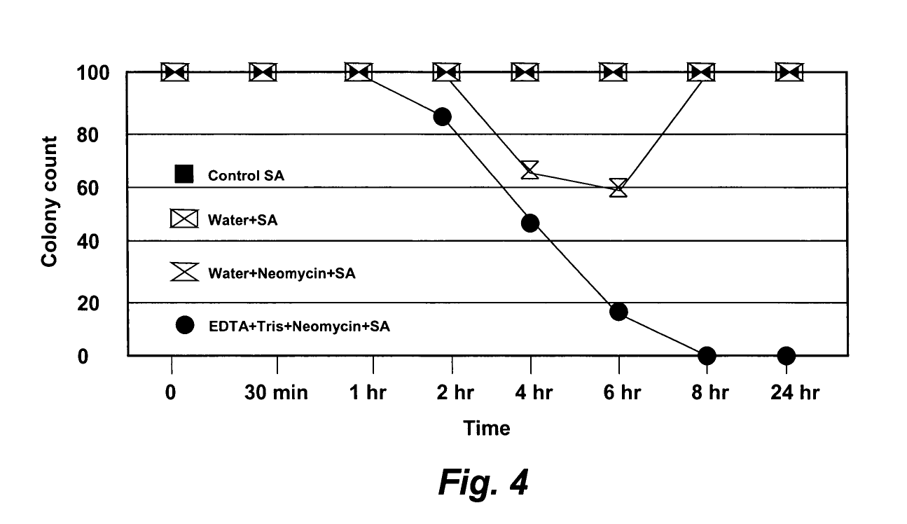

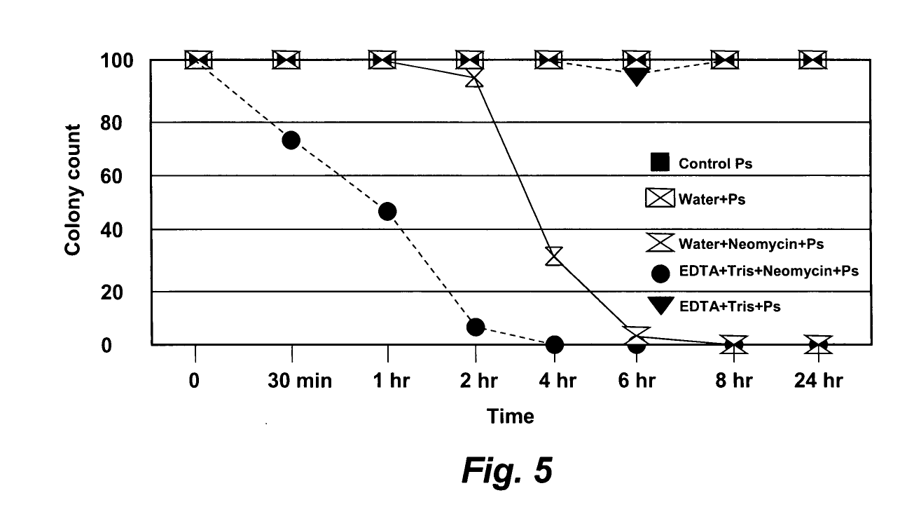

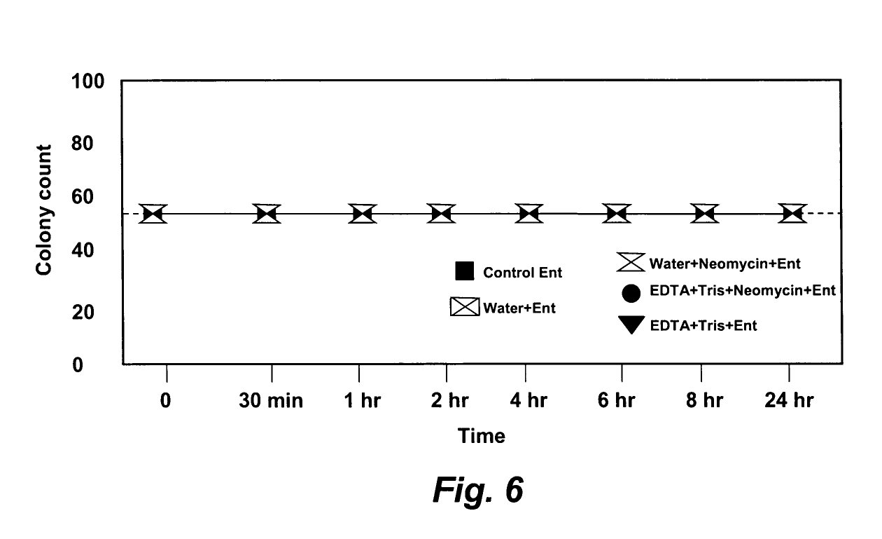

In the in vitro gauze-point-contactor study, the potentation effect was seen for EDTA-Tris-neomycin reactions with Ps. aeruginosa and Staph. aureus. These reactions are illustrated in FIGS. 4 and 5. When the same combinations of EDTA-Tris and neomycin were reacted with Ent. faecalis, no antibacterial activity was noted at these concentrations as shown in FIG. 6.

### EXAMPLE 6

### Formulations of Wash solutions

### Percent Cocamidopropyl Betaine

| | 2.5 | 5 | 10 | 15 | 20 | 25 |
|---|---|---|---|---|---|---|
| 0.5 M EDTA | 16 μl | 16 μl | 16 μl | 16 μl | 16 μl | 16 μl |
| 1.0 M Tris | 20 μl | 20 μl | 20 μl | 20 μl | 20 μl | 20 μl |
| Deionized | 939 μl | 914 μl | 864 μl | 814 μl | 764 μl | 714 μl |
| water | | | | | | |
| Cocamido- | 25 μl | 50 μl | 100 μl | 150 μl | 200 μl | 250 μl |
| propyl betaine | | | | | | |

Total volume = 1.0 ml of prewash.
Final concentrations of EDTA and Tris are 8 mM EDTA and 20 mM Tris.

### EXAMPLE 7

### Use of Tris-EDTA-Cocamidopropyl Betaine as an Animal ear skin Cleanser

A solution containing cocamidopropyl betaine (5%-15%), 8 mM EDTA and 20 mM Tris, in deionized water, was used by five clinicians to clean the ears of more than 20 dogs. The severity of exudates (ceruminous otitis) varied from mild to severe. The treated external ear canal was filled with warm ear cleaner and gently messaged during a 1-5 minute period. The external ear was then washed with warm saline. The cleaning procedure was repeated as needed to remove all visible exudate and prepare the ear for antimicrobial treatment.

All clinicians reported that the solution was effective, caused minimal to no discomfort (even in severely inflamed ears) and did not worsen the inflammatory process. In addition, the solution was used with no apparent side effects, in several dogs that were subsequently shown to have ruptured tympanic membranes.

### EXAMPLE 8

### Tris-EDTA with Cocamidopropyl Betaine as an Antimicrobial Composition

Initially, an 8 mM Tris-20 mM EDTA solution (lx formulation) with 2.5%, 5%, 10%, 15%, 20%, or 25% cocamidopropyl betaine was prepared and compared for cleaning efficacy. The solution containing 10% cocamidopropyl betaine was selected for further testing.

The formulation was tested against Pseudomonas aeruginosa, Proteus mirabilis, Eshcericiha coli, Staphylococcus intermedius, Malassezia pachydermatis, and Candida albicans.

TABLE 4
MIC and MBC data for cocamidopropyl betaine alone

| Organism | MIC^{a} | MBC^{a} |
|---|---|---|
| P. aeruginosa | 25 | 50 |
| P. mirabilis | 25 | 50 |
| E. coli | 25 | 50 |
| S. intermedius | 0.1 | 0.1 |
| M. pachydermatis | 0.1 | 0.3 |
| C. albicans | 0.1 | 0.3 |

^{a}percent of stock solution

TABLE 5
MIC and MBC data for Tris-EDTA alone

| Organism | MIC^{b} | MBC^{b} |
|---|---|---|
| P. aeruginosa | 100 | >100 (125% or 1.25x) |
| P. mirabilis | >100 (500% or 5x) | >100 (1000% or 10x) |
| E. coli | >100 (500% or 5x) | >100 (500% or 5x) |
| S. intermedius | 12.5 | 25 |
| M. pachydermatis | 3.9 | 7.8 |
| C. albicans | 7.8 | 62.5 |

^{b}percent of 1x formulation

TABLE 6
MIC and MBC data for 1xTris-EDTA formulation
with 10% cocamidopropyl betaine)

| Organism | MIC^{c} | MBC^{c} |
|---|---|---|
| P. aeruginosa | 12.5 | 100 |
| P. mirabilis | 25 | >100 (125% or 1.25x) |
| E. coli | 6.25 | 6.25 |
| S. intermedius | <0.2 | <0.2 |
| M. pachydermatis | 3.9 | 3.9 |
| C. albicans | <0.2 | <0.2 |

^{c}percent of 1x formulation

The only organism not killed, even though its growth was inhibited, by the test cleaner formulation was Proteus mirabilis. Based on MIC data, there is synergistic effects for the 1× formulation with 10% cocamidopropyl betaine reacting against Pseudomonas aeruginosa, Proteus mirabilis, and Escherichia coli.

What is claimed is:

1. A skin cleanser comprising a chelating agent, a pH buffer, and a detergent, wherein the amounts of the chelating agent and the detergent relative to each other are selected to allow the chelating agent and the detergent to synergistically cooperate to enhance antimicrobial activity of the skin cleanser when in aqueous solution.

2. The skin cleanser of claim 1, further comprising a carrier.

3. The skin cleanser of claim 1, further comprising an antimicrobial agent, or a combination of antimicrobial agents.

4. The skin cleanser of claim 1, further comprising an anti-inflammatory agent.

5. The skin cleanser of claim 1, wherein the chelating agent is selected from the group consisting of ethylenediamenetetracetic acid (EDTA), triethylene tetramine dihydrochloride (TRIEN), ethylene glycol-bis (beta-aminoethyl ether)-N, N, N′, N′-tetracetic acid (EGTA), diethylenetriamin-pentaacetic acid (DPTA), triethylenetetramine hexaacetic acid (TTHA), deferoxamine, Dimercaprol, edetate calcium disodium, zinc citrate, penicilamine succimer and Editronate.

6. The skin cleanser of claim 1, wherein the chelating agent is ethylenediamenetetracetic acid (EDTA).

7. The skin cleanser of claim 1, wherein the pH buffer is Tris (hydroxymethyl) aminomethane base.

8. The skin cleanser of claim 1, wherein the detergent is cocamidopropyl betaine.

9. The skin cleanser of claim 2, wherein the carrier is an aqueous carrier.

10. The skin cleanser of claim 4, wherein the anti-inflammatory agent is dexamethasone.

11. The skin cleanser of claim 3, wherein the antimicrobial agent is a β-lactam, an aminoglycoside, a vancomycin, a bacitracin, a macrolide, an erythromycin, a lincosamide, a chloramphenicol, a tetracycline, a gentamicin, an amphotericin, a cefazolin, a clindamycin, a mupirocin, a nalidixic acid, a sulfonamide and trimethoprim, a streptomycin, a rifampicin, a metronidazole, a quinolone, a novobiocin, a polymixin, a gramicidin, clomtrimazole, miconazole, natamycin, amphotericin B, cuprimycin, enilconazole, fluconazole, haloprogin, ketoconazole, nystatin or tolnaftate, or a mixture thereof.

12. The skin cleanser of claim 2, wherein the concentration of the chelating agent is between about 5 mM and about 250 mM.

13. The skin cleanser of claim 2, wherein the concentration of the pH buffer is between about 5 mM and about 250 mM, and the cleanser has a pH of between about 6.0 and about 9.0.

14. The skin cleanser of claim 2, wherein the concentration of the detergent is between about 1% and 30%, by volume.

15. The skin cleanser of claim 2, further comprising an antimicrobial agent having a concentration between about 1 μg/ml and about 5 mg/ml.

16. The skin cleanser of claim 2, comprising about 8 mM of a chelating agent, about 20 mM of a pH buffer and about 10%, by volume, detergent.

17. The skin cleanser of claim 16 having a pH from about 6.0 to about 9.0.

18 The skin cleanser of claim 16, wherein the chelating agent is ethylenediamenetetracetic acid (EDTA), the pH buffer is Tris (hydroxymethyl) aminomethane base and the detergent is cocamidopropyl betaine.

19. The skin cleanser of claim 1, further comprising a stabilizer.

20. The skin cleanser of claim 19, wherein the stabilizer is scorbic acid.

21. The skin cleanser of claim 1, further comprising a colorant.

22. The skin cleanser of claim 1, further comprising a perfume.

23. A method of cleansing a surface comprising the steps of:
applying a skin cleanser to a surface, said skin cleanser comprising a chelating agent, a pH buffer, a detergent and a carrier, wherein the skin cleanser has antimicrobial activity, and wherein the amounts of the chelating agent and the detergent are selected to allow the chelating agent and the detergent to synergistically enhance the antimicrobial activity of the skin cleanser;
leaving the skin cleanser on the surface for sufficient time to loosen contaminants on said surface, whereby the chelating agent synergistically cooperates with the detergent to reduce the amount of contaminants on the surface; and
removing the skin cleanser from the surface.

24. The method of claim 23, wherein the skin cleanser is applied to the skin surface of an animal.

25. The method of claim 24, wherein the skin cleanser is applied to the external surface of the ear of the animal.

26. The method of claim 23, wherein the skin cleanser is applied to an inanimate surface.

27. The method of claim 23, wherein the skin cleanser is applied to the hair of an animal.

28. The method of claim 23, further comprising the step of applying an antimicrobial agent to the surface.

29. The method of claim 23, wherein the skin cleanser further comprises an antimicrobial agent.

30 The method of claim 28, wherein the antimicrobial agent is applied after the skin cleanser, and wherein the antimicrobial agent is applied with a chelating agent, and wherein the chelating agent synergistically cooperates with the antimicrobial agent to reduce a microbial population of the skin surface

31. The method of claim 23, wherein the chelating agent is selected from the group consisting of ethylenediamenetetracetic acid (EDTA), triethylene tetramine dihydrochloride (TRIEN), ethylene glycol-bis (beta-aminoethyl ether)-N, N, N′, N′-tetracetic acid (EGTA), diethylenetriamin-pentaacetic acid (DPTA), triethylenetetramine hexaacetic acid (TTHA), deferoxamine, Dimercaprol, edetate calcium disodium, zinc citrate, penicilamine succimer and Editronate, and has a concentration between about 1 mM and about 250 mM.

32. The method of claim 30, wherein the chelating agent applied with the antimicrobial agent is about 8 mM EDTA.

33. The method of claim 23, wherein the pH buffer is Tris (hydroxymethyl) aminomethane base having a concentration between about 5 mM and about 250 mM.

34. The method of claim 33, wherein the pH buffer is about 20 mM Tris.

35. The method of claim 23, wherein the detergent is cocamidopropyl betaine having a concentration of about 10%, by volume.

36. The method of claim 23, wherein the skin cleanser comprises about 8 mM EDTA, about 20 mM Tris and about 10% cocamidopropyl betaine.

37. The method of claim 28, wherein the antimicrobial agent is a β-lactam, an aminoglycoside, a vancomycin, a bacitracin, a macrolide, an erythromycin, a lincosamide, a chloramphenicol, a tetracycline, a gentamicin, an amphotericin, a cefazolin, a clindamycin, a mupirocin, a nalidixic acid, a sulfonamide and trimethoprim, a streptomycin, a rifampicin, a metronidazole, a quinolone, a novobiocin, a polymixin, a gramicidin, clomtrimazole, miconazole, natamycin, amphotericin B, cuprimycin, enilconazole, fluconazole, haloprogin, ketoconazole, nystatin or tolnaftate, or a mixture thereof.

38. The method of claim 23, wherein the skin cleanser comprises about 8 mM EDTA, about 20 mM Tris, about 10% cocamidopropyl betaine and an antimicrobial agent having a concentration of between about 11 g/ml and about 5 mg/ml.

39. The method of claim 23, wherein the skin cleanser is added to a medical dressing before contacting the skin surface.

40. A kit comprising a vessel containing a skin cleanser comprising a chelating agent, a pH buffer, and a detergent, wherein the amounts of the chelating agent and the detergent relative to each other are selected to allow the chelating agent and the detergent to synergistically cooperate to enhance antimicrobial activity of the skin cleanser when in aqueous solution, and packaging material comprising instructions for preparing the skin cleanser as an aqueous solution and contacting said cleanser with a skin surface.

41. The kit according to claim 40, wherein the skin cleanser further comprises an antimicrobial agent, and further comprises instructions for using the antimicrobial agent with the skin cleanser to clean and sanitize a skin surface.

42. The kit according to claim 40, further comprising instructions for using the skin cleanser to reduce otitis externa of an animal or human.

43. The kit according to claim 40, further comprising a medical dressing configured to receive the cleanser and instructions for using the medical dressing to deliver the cleanser to the skin surface of an animal or human.
